(19) United States
(12) Patent Application Publication
(10) Pub. No.: US 2012/0050515 A1
(43) Pub. Date: Mar. 1, 2012

(54) IMAGE PROCESSING APPARATUS AND IMAGE PROCESSING METHOD

(75) Inventors: Masao Shikaumi, Tokyo (JP); Hiroshi Hara, Machida-shi (JP)

(73) Assignee: CANON KABUSHIKI KAISHA, Tokyo (JP)

(21) Appl. No.: 13/208,898

(22) Filed: Aug. 12, 2011

(30) Foreign Application Priority Data
Aug. 31, 2010 (JP) 2010-194718

(51) Int. Cl. A61B 3/14; H04N 7/18

(52) U.S. Cl. 348/78; 351/206; 348/E07.085

(57) ABSTRACT

A fundus camera, having a first light source for observing an object, a second light source for photographing the object and an image pickup element, determines a light intensity of the second light source based on first image data obtained by illuminating an eye to be examined through the first light source and by forming an image on the image pickup element using light reflected from the eye to be examined. A computer analyzes second image data obtained by illuminating the eye to be examined through the second light source with the light intensity determined by the fundus camera and by forming an image on the image pickup element using light reflected from the eye to be examined. The computer controls brightness correction processing of the second image data based on the analysis result and information relating to a time of photographing the second image data.

### BACKGROUND OF THE INVENTION

1. Field of the Invention

The present invention relates to technology that determines a light intensity of a photographing light source based on observation image data that is output as a result of a light that is radiated by an observation light source being reflected from an eye to be examined and forming an image on an image pickup unit.

2. Description of the Related Art

A fundus camera has been conventionally used for fundus oculi examination, diabetes testing and other purposes. With such a conventional fundus camera, a fundus oculi is illuminated with visible light or infrared light for positioning and focusing, and an image of the fundus oculi is captured using a visible light source such as a strobe light. The reflectance of the fundus oculi differs according to the race of an individual. In order to suppress such differences in the reflectance of the fundus oculi caused by racial differences, Japanese Patent No. 2974751 (JP04-150831A) discloses technology of a fundus camera that automatically adjusts a photographing light intensity.

The technology disclosed in Japanese Patent No. 2974751 irradiates an infrared light for photometric measurement at the fundus oculi when performing observation, measures a reflected light from the fundus oculi, and adjusts a photographing light intensity. In this case, a visible light such as a strobe light or a white LED is used for photographing. However, even in the case of a fundus oculi of the same individual, the reflectance of the fundus oculi with respect to an infrared light and the reflectance of the fundus oculi with respect to a visible light are different. The technology disclosed in Japanese Patent No. 2974751 can not suppress differences in reflectance that are caused by such differences in the wavelengths used. Furthermore, recently, some digital cameras are also known that are equipped with a function (automatic light adjustment function) that allows the camera to automatically adjust the brightness of an image if the image is dark after photographing.

### SUMMARY OF THE INVENTION

In this case, a captured range in an image of a general photographing scene is different to a captured range when photographing a fundus oculi image. Further, information relating to a time of photographing by a fundus camera is not sent to a digital camera that is attached to the fundus camera. It is therefore difficult for an in-built automatic light adjustment function of the digital camera to adjust the brightness of an image based on differences in the reflectance of the fundus oculi caused by racial or individual differences.

Furthermore, if the photographer increases or decreases the photographing light intensity on the fundus camera side when photographing in order to photograph a brighter image or darker image according to the preference of the photographer, that information is not reflected in operations performed on the digital camera side. Consequently, the automatic light adjustment function in the digital camera causes the image to have the same uniform brightness.

An object of the present invention is to realize an automatic light adjustment function that adjusts a brightness of an image on the basis of differences in the reflectance of a fundus oculi that are due to racial or individual differences, and also reflect a preference of a photographer with regard to exposure.

In order to achieve the object discussed above, the present invention provides with an image processing apparatus for connection with an image pickup apparatus having a first light source for observing an object, a second light source for photographing the object and an image pickup unit, wherein the image pick up apparatus determines a light intensity of the second light source based on first image data obtained by illuminating an eye to be examined through the first light source and by forming an image on the image pickup unit using light reflected from the eye to be examined, the image processing apparatus comprising: an analysis unit that analyzes second image data obtained by illuminating the eye to be examined through the second light source with the light intensity determined by the image pickup apparatus and by forming an image on the image pickup unit using light reflected from the eye to be examined; and a control unit that controls brightness correction processing with respect to the second image data based on an analysis result obtained by the analysis unit and information relating to a time of photographing the second image data.

According to the present invention, it is possible to realize an automatic light adjustment function that adjusts a brightness of an image on the basis of differences in the reflectance of a fundus oculi that are due to racial or individual differences, and also reflect a preference of a photographer with regard to exposure.

Further features of the present invention will become apparent from the following description of exemplary embodiments with reference to the attached drawings.

### BRIEF DESCRIPTION OF THE DRAWINGS

FIG. 1 is a view that illustrates a configuration of an ophthalmologic photographing apparatus according to a first to third embodiment of the present invention.

FIG. 2 is a view for describing photometry in a normal state.

FIG. 3 is a flowchart that illustrates brightness correction processing that is executed by a computer.

FIG. 4 is a view for describing photometry in an anterior ocular segment photographing state.

FIG. 5 is a view for describing photometry in a small pupil photographing state.

FIG. 6 is a view for describing photometry in a variable magnification photographing state.

FIGS. 7A and 7B are views for describing photometry of left and right eyes.

FIG. 8 is a view for describing vignetting correction.

FIG. 9 is a view that illustrates a configuration of an ophthalmologic photographing apparatus according to a fourth embodiment of the present invention.

FIG. 10 is a view for describing photometry in a mydriatic photographing state.

### DESCRIPTION OF THE EMBODIMENTS

Preferred embodiments of the present invention will now be described in detail in accordance with the accompanying drawings.

Exemplary embodiments to which the present invention is applied are described in detail below with reference to the attached drawings.

### First Embodiment

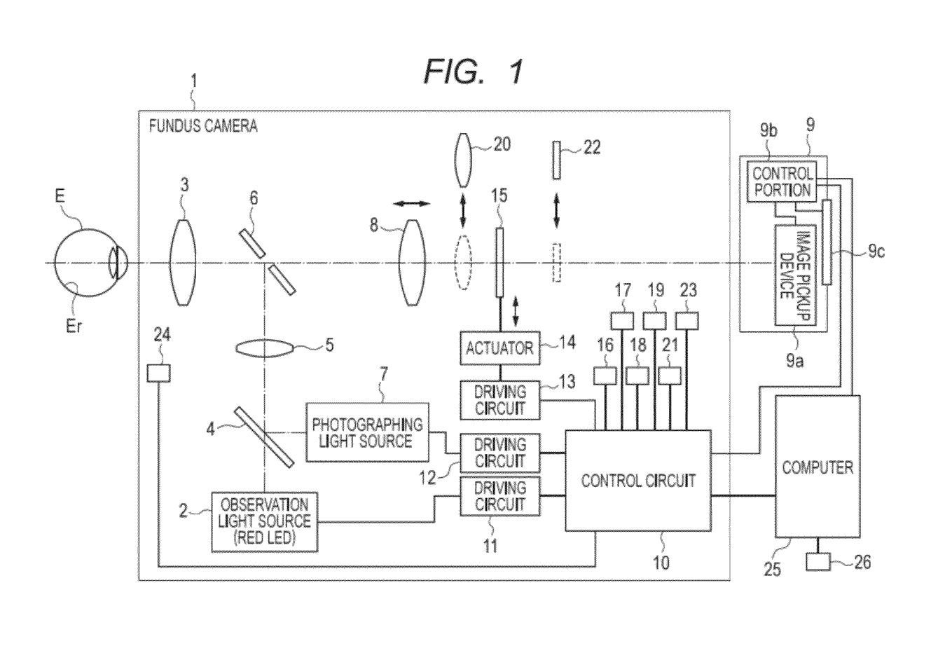

First, a first embodiment of the present invention is described. FIG. 1 is a view that illustrates a configuration of an ophthalmologic photographing apparatus according to the first embodiment of the present invention. A fundus camera 1 according to the present embodiment is a non-mydriatic fundus camera that is placed in front of an eye to be examined E. The fundus camera body 1 includes an observation illumination optical system that extends from an observation light source 2 to an objective lens 3. The observation light source 2 irradiates an infrared light, and includes, for example, an infrared LED. The objective lens 3 is placed so as to face the eye to be examined E. The observation illumination optical system includes the observation light source 2, a dichroic mirror 4, a relay lens 5, and a perforated mirror 6 that are arranged in that order. The fundus camera body also includes a photographing light source 7 as a photographing illumination optical system disposed in an incident direction of the dichroic mirror 4. The photographing light source 7 includes a visible light source such as a xenon tube or a white LED. In this connection, the observation light source 2 constitutes an application example of a first light source, and the photographing light source 7 constitutes an application example of a second light source.

A focusing lens 8 as a photographing optical system is disposed behind the perforated mirror 6. The focusing lens 8 moves in a direction of an optical axis to adjust focus. A digital single-lens reflex camera 9 (hereunder, referred to as “digital camera 9”) that is a photographing unit is removably attached to the fundus camera body 1 on an extension of the optical axis of the focusing lens 8.

The digital camera 9 includes an image pickup device 9a that converts a formed optical image into an electrical signal. An output of the image pickup device 9a is connected to a control portion 9b in the digital camera 9. A liquid crystal display 9c that serves as a display portion is provided on the back of the digital camera 9. An output of the control portion 9b is connected to the liquid crystal display 9c. To provide the digital camera 9 with near-infrared sensitivity, an optical member having infrared cut-off characteristics that is conventionally disposed on the front face of the image pickup device 9a has been removed.

The fundus camera body 1 further includes a control circuit 10. Outputs of the control circuit 10 are connected to the observation light source 2 and the photographing light source 7 via driving circuits 11 and 12, respectively. The control circuit 10 is also connected to the control portion 9b in the digital camera 9 and to a release switch 16 that is provided on the fundus camera body 1.

The control circuit 10 is also connected to an actuator 14 via a driving circuit 13. When the actuator 14 is driven, the actuator 14 inserts an infrared cut-off filter 15 into the photographing optical path or withdraws the infrared cut-off filter 15 therefrom. The infrared cut-off filter 15 has the same characteristics as the infrared cut-off characteristics of the optical member that has been removed from the image pickup device 9a of the digital camera 9. At a time of observation, observation of a fundus oculi image by means of the digital camera 9 is enabled by withdrawing the infrared cut-off filter 15 to a position outside the photographing optical path. At a time of photographing, by inserting the infrared cut-off filter into the photographing optical path, spectral characteristics that are equivalent to those of a general digital camera are obtained by means of the digital camera 9 and the infrared cut-off filter 15.

Switches including a light adjustment correction switch 17, an automatic light adjustment on/off switch 18, an anterior ocular segment photographing switch 19, a small pupil photographing switch 21, a variable magnification photographing switch 23, and a left/right eye detection switch 24 are also connected to the control circuit 10.

The present embodiment has an automatic light adjustment effect that obtains an image of a predetermined brightness by detecting a luminance of a fundus oculi image that is illuminated by infrared light from the observation light source 2 at a time of observation, and automatically adjusting a photographing light intensity at a time of photographing with visible light from the photographing light source 7.

The light adjustment correction switch 17 is a switch for performing a light adjustment correction so that the result of automatic light adjustment is a preferred brightness of the photographer. For example, the light adjustment correction switch 17 is a seesaw switch that is adapted so as to correct the brightness by an amount of +0.3V each time one side of the switch is pressed, and correct the brightness by an amount of −0.3V each time the opposite side of the switch is pressed. The automatic light adjustment on/off switch 18 enables (turns on) or disables (turns off) the above described automatic light adjustment function.

The anterior ocular segment photographing switch 19 is used to switch between a fundus oculi photographing mode and an anterior ocular segment photographing mode. When the apparatus enters the anterior ocular segment photographing mode as a result of operating the anterior ocular segment photographing switch 19, an anterior ocular photographing optical system 20 is inserted at a position that is partway along the photographing optical system to thereby obtain a photographing angle of view that is most appropriate for anterior ocular segment photographing, and the anterior ocular segment of the examinee can be photographed.

The small pupil photographing switch 21 is used to switch between normal photographing and small pupil photographing. When the small pupil photographing switch 21 is operated, a baffle for small pupil photographing 22 is inserted into the photographing optical path. Although only the baffle for small pupil photographing 22 is schematically illustrated in FIG. 1, switching of a required optical system (not shown) such as switching of a diaphragm or a lens system may also be performed. When performing small pupil photographing, since the influence of flare is liable to appear, the photographing angle of view is limited to a range that is narrower than when performing normal photographing.

The variable magnification photographing switch is used to switch to a variable magnification photographing mode. The variable magnification photographing mode according to the present embodiment is configured to perform digital variable magnification photographing at two-fold magnification by digitally trimming and cutting out half of a center part of image data. The left/right eye detection switch 24 is mounted on an unshown stage portion of the fundus camera body 1, and is used to detect whether the left eye or the right eye of the examinee is being photographed.

A computer 25 is provided outside the fundus camera body 1. The computer 25 is connected to the control portion 9b of the digital camera 9 and the control circuit 10 of the fundus camera body 1 through a USB or a serial port.

An information input section 26 is connected to the computer 25. The information input section 26 is used for inputting an ID number and personal information of the examinee as well as information regarding a disease or the like of the examinee prior to photographing. When the release switch 16 is pressed, the control circuit 10 sends a release signal to the control portion 9b of the digital camera 9. Upon receipt of the release signal, the digital camera 9 performs a photographing operation.

After photographing, the control portion 9b of the digital camera 9 sends the captured image data to the computer 25. Various kinds of image processing such as the addition of data for an electronic mask image and vignetting correction, including brightness correction, which are described later are performed at the computer 25 based on the captured image data.

Further, information regarding a light adjustment correction value at a time of photographing, as well as the on/off state of the automatic light adjustment function, whether or not the photographing is anterior ocular segment photographing, fundus oculi photographing, or small pupil photographing, whether or not the photographing is variable magnification photographing, and whether the left eye or the right eye has been photographed is sent to the computer 25 from the control circuit 10.

Recently, an increasing number of models of digital cameras are equipped with a live view function. The term “live view” refers to a function in which, with the shutter opened by retracting a quick-return mirror included in the digital camera 9, images formed on the image pickup device 9a are successively read to continuously display image data of the images on the liquid crystal display 9c on the back of the camera. According to the present embodiment, the live view function of the digital camera 9 is used at the time of observation, and a photometry result obtained with infrared light at the time of observation is used for light adjustment control when photographing.

Next, photometric operations that are performed on the fundus camera body 1 side are described. Infrared light that is reflected from the fundus oculi of the eye to be examined while in a live view state forms an image on the image pickup device 9a. The corresponding image data is read by the control portion 9b and displayed on the liquid crystal display 9c, and is also used to perform a photometric calculation.

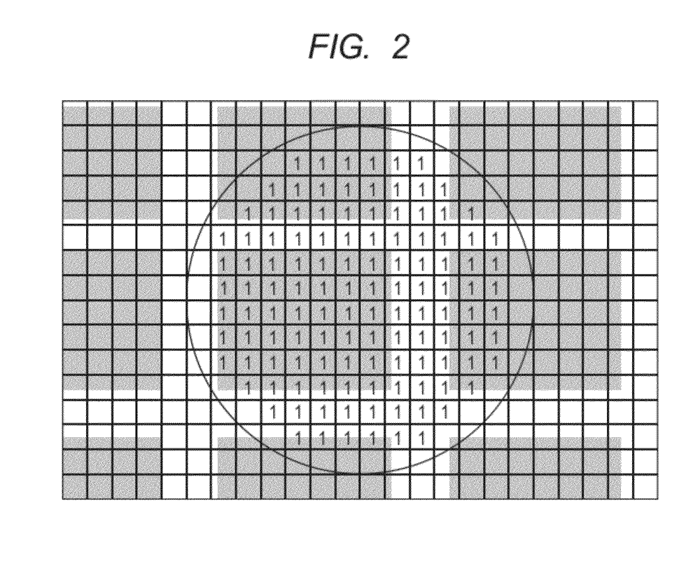

FIG. 2 is a view for describing photometry in a normal state. The live view image data is split into small blocks as shown in FIG. 2. According to the first embodiment, it is assumed that the live view image data is split into small blocks that are arranged in a 16 (length)×24 (breadth) block layout. A fundus oculi image is captured inside a range indicated by the circle in FIG. 2 that is in the center of the image pickup device of the digital camera 9, and the outside thereof is masked. Further, a mean infrared luminance value inside the small blocks into which the live view image data is split is calculated. A luminance value Y can be determined by the following luminance conversion equation using R, G, and B pixel values of the image.

Y=0.299R+0.587G+0.114B

According to the first embodiment, the digital camera 9 that is used has been modified so as to be sensitive to infrared light. Consequently, the output of each of the R, G, and B pixels deviates from normal spectral characteristics. Therefore, for example, an infrared luminance value may be determined using a value of only a G pixel or an R pixel, without depending on the above luminance conversion equation.

Next, the overall photometric value is determined based on the infrared luminance value of each small block. Since the fundus oculi image is only captured inside the circle shown in FIG. 2 as described above, only the blocks that are present inside the circle may be evaluated. Thus, in FIG. 2, only the blocks which contain the numeral “1” are extracted, and the infrared luminance values thereof are averaged to obtain an overall photometric value.

Although the output of the image pickup device 9a is output as a value that is linearly proportional to the light intensity, in that state the output of a bright portion will be high and will influence a value that is output. Hence, to facilitate the photometric calculation, the logarithm to base 2 of the mean infrared luminance value of the small blocks may be taken, and the mean value thereof obtained and employed as a photometric value. Automatic light adjustment is performed by controlling a light emission quantity or a light emission time period of the photographing light source 7 at the time of photographing based on the relationship between the infrared photometric value and a visible photographing light that is previously known.

Although there is the difference that infrared light is used when performing photometry and visible light is used when photographing, the influence of pupillary diameter is equal with respect to infrared light and visible light. Hence, fundamentally, photometry can be performed with infrared light, and the photometry result can be reflected in visible light photographing.

Although FIG. 2 illustrates photometry in a normal state, depending on the mode that is set in the fundus camera body 1, the range of blocks used for photometric evaluation can be changed or the weightings of the values of the respective blocks can be changed. Further, when light adjustment correction is being applied by operating the light adjustment correction switch 17, a light emission quantity or a light emission time period when photographing can be controlled in accordance with the light adjustment correction amount, and a predetermined correction effect can be obtained.

However, there are individual differences between the infrared reflectance and the visible light reflectance of the fundus oculi, and those differences cannot be absorbed only by the automatic light adjustment operation of the fundus camera body 1. Therefore, the image data after photographing is sent to the computer 25, and brightness correction is performed at the same time as application of an electronic mask or vignetting correction to thereby absorb individual differences with respect to reflectance of infrared light and visible light.

Next, brightness correction processing of the computer 25 is described. Evaluation of the brightness of image data at the computer 25 is carried out in the same manner as a calculation when performing infrared photometry at the fundus camera body 1.

As described above with reference to FIG. 2, the computer 25 splits image data into small blocks, calculates mean luminance values, and determines a mean luminance value of the image data with respect to the required blocks. Subsequently, the computer 25 calculates a deviation between the mean luminance value and a target, and if the deviation is greater than a predetermined value, the computer 25 corrects the brightness to bring the mean luminance value closer to the target value by performing known image processing such as changing the overall brightness, correcting the contrast, or performing histogram processing.

When determining a mean luminance value for each small block, the computer 25 can determine the mean luminance value based on thumbnail image data that is small image data that the digital camera 9 creates at the time of photographing. Further, when it is desired to perform a calculation with a greater level of detail, the computer 25 can perform the calculation on the basis of so-called RAW image data that is the image data before development that is sent from the digital camera 9.

When performing brightness correction is at the computer 25, it is necessary to know the conditions under which photographing was performed when the image was captured. Information showing the conditions at the time of photographing (hereunder, referred to as “time-of-photographing information”) is sent to the computer 25 together with the image data at the time of photographing by the control circuit 10 inside the fundus camera body 1.

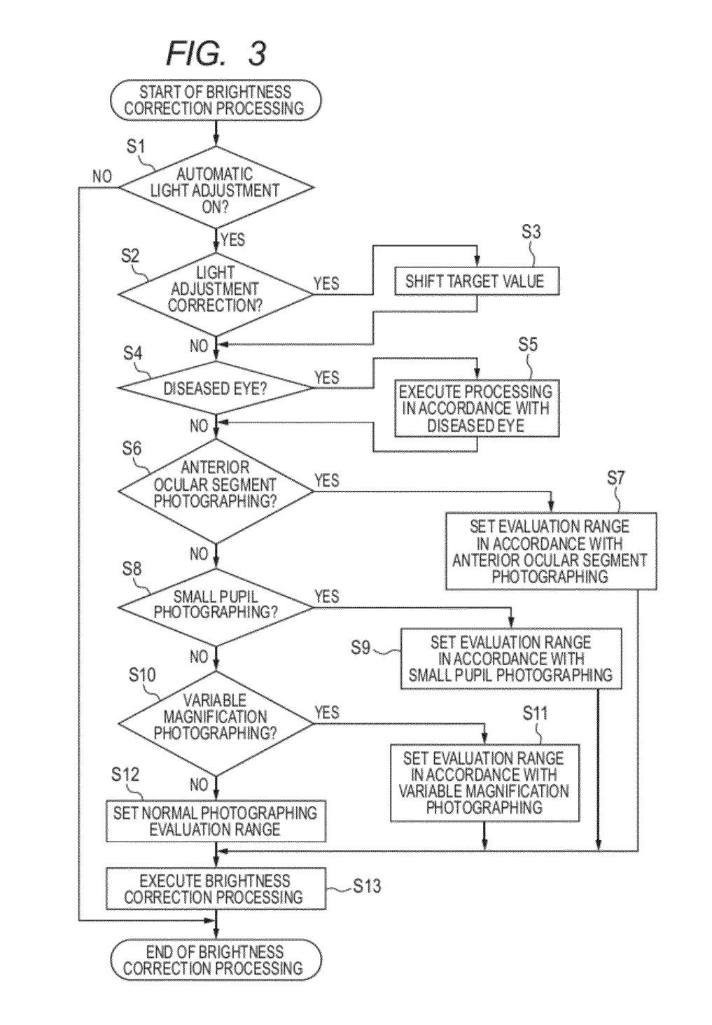

FIG. 3 is a flowchart that illustrates the brightness correction processing performed by the computer 25. This flowchart is executed when brightness correction processing starts.

The computer 25 refers to the time-of-photographing information sent by the control circuit 10, and determines whether the automatic light adjustment function was on or off when photographing was performed (step S1). If the automatic light adjustment function was off, the computer 25 ends the brightness correction processing. In contrast, if the automatic light adjustment function was on when photographing was performed, the computer 25 determines whether or not light adjustment correction is applied to the image (step S2). If light adjustment correction is applied, the computer 25 shifts the target value of brightness correction according to the light adjustment correction value (step S3).

Subsequently, the computer 25 refers to diseased eye information of the examinee that has been input through the information input section 26 (step S4). If the diseased eye information indicates that the examinee has a diseased eye, the computer 25 performs processing in accordance with the disease (step S5). For example, in the case of a cataract, the computer 25 makes the brightness of image data obtained by visible light photographing brighter in comparison to an infrared photometric value. Since the image becomes darker than a normal cataract image when brightness correction is applied thereto, in the case of a cataract the computer 25 shifts the target value to obtain a brighter value. Depending on the disease type, for some diseases the brightness correction processing may be applied in the normal manner. The computer 25 changes the determination regarding the manner in which processing is to be performed in accordance with the diseased eye information of the examinee.

Next, the computer 25 determines whether or not anterior ocular segment photographing has been performed (step S6). If anterior ocular segment photographing has been performed, the computer 25 sets an evaluation range for brightness correction that corresponds to the anterior ocular segment photographing (step S7).

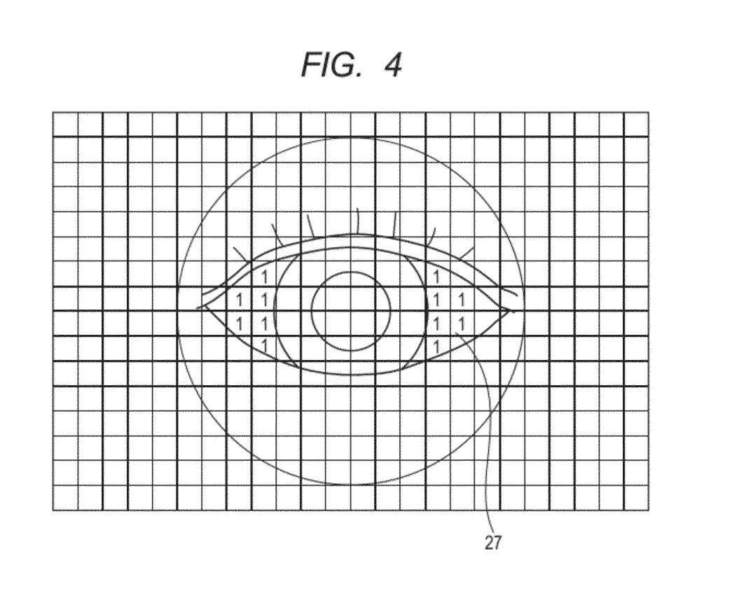

FIG. 4 is a view for describing photometry in an anterior ocular segment photographing state. When anterior ocular segment photographing is performed, a captured image includes skin and the iris. However, it is difficult to determine the brightness of the skin or an iris part because there are both racial differences and individual differences. Therefore the computer 25 evaluates a white of the eye (sclera) portion and performs the brightness correction.

A sclera 27 illustrated in FIG. 4 is used for evaluation in brightness correction of only the blocks that contain the numeral “1” among the small blocks. Further, in order to make the brightness match in the whitish sclera portion, the computer 25 also changes the brightness target value in comparison to the case of the fundus oculi image. Subsequently, the computer 25 advances to step S13 to perform brightness correction processing.

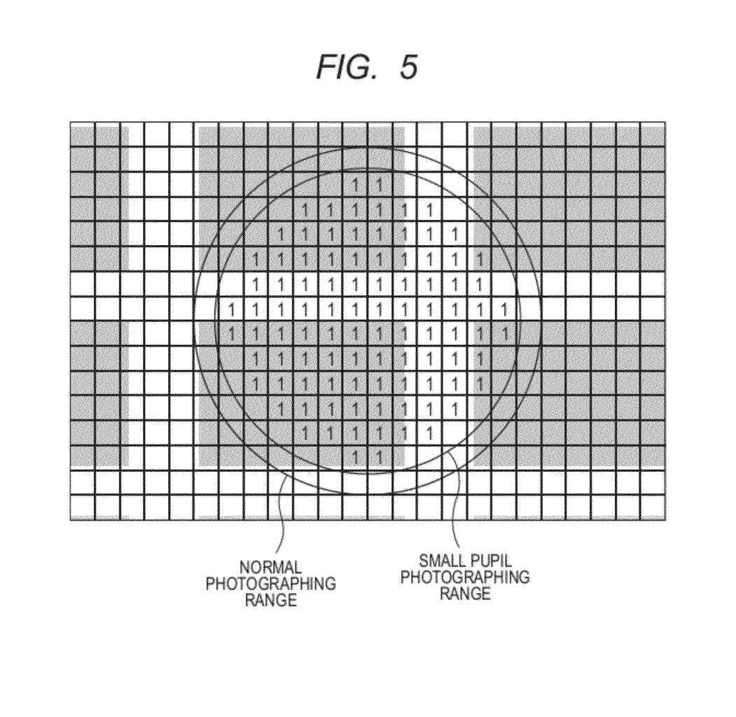

If anterior ocular segment photographing has not been performed, the computer 25 determines whether or not small pupil photographing has been performed (step S8). If small pupil photographing has been performed, the computer 25 sets the evaluation range of the brightness correction in accordance with small pupil photographing (step S9). FIG. 5 is a view for describing photometry in a small pupil photographing state. In small pupil photographing, the photographing range is smaller than in normal photographing. Hence, a region used for evaluation (blocks which contain the numeral “1” in FIG. 5) is also smaller than at a time of normal photographing. After setting the evaluation range for small pupil photographing, the computer 25 advances to step S13 to perform brightness correction processing.

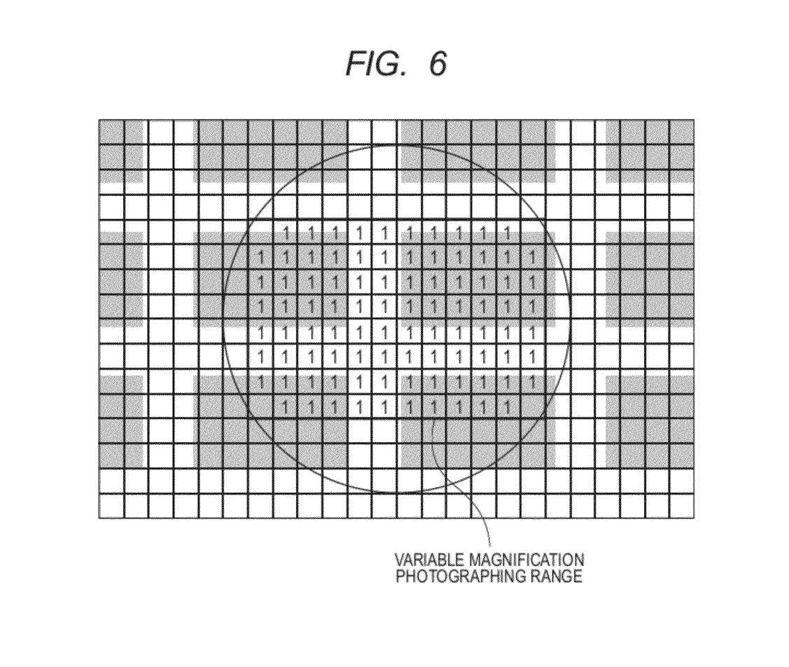

Next, the computer 25 determines whether or not variable magnification photographing has been performed (step S10). If variable magnification photographing has been performed, the computer 25 sets the evaluation range of the brightness correction in accordance with variable magnification photographing (step S11). FIG. 6 is a view for describing photometry in a variable magnification photographing state. The variable magnification photographing according to the present embodiment is two-fold magnification photographing, and the image data in an area surrounded by a thick line in FIG. 6 is trimmed.

Hence, the evaluation range for the brightness correction is the blocks which contain the numeral “1” in FIG. 6. After setting the evaluation range for variable magnification photographing, the computer 25 advances to step S13 to perform brightness correction processing.

In contrast, if it is determined in step S10 that variable magnification photographing has not been performed, since normal photographing has been performed the computer 25 sets the evaluation range for normal photographing that is described above using FIG. 2 (step S12), and advances to step S13 to perform brightness correction processing for the image data. As described above, the brightness correction processing is performed by known image processing such as changing the overall brightness, correcting the contrast, or performing histogram processing.

As described in the foregoing, according to the first embodiment, the fundus camera 1 uses observation image data that is output from the digital camera 9 to determine the light intensity of the photographing light source 7 to be used when photographing the eye to be examined E. The computer 25 analyzes a photographed image that has been photographed using the photographing light source 2 that emits a quantity of light in accordance with the determined light intensity. The computer 25 controls brightness correction processing with respect to the photographed image data based on the analysis result and information relating to the time of photographing the photographed image data. Thus, according to the present embodiment, an ophthalmologic photographing apparatus can be obtained that is equipped with a light adjustment function that is not dependent on racial or individual differences, and that can reflect a preference of a photographer regarding exposure. Note that observation image data constitutes an application example of first image data, and photographed image data constitutes an application example of second image data.

### Second Embodiment

Next, a second embodiment of the present invention is described. The configuration of an ophthalmologic photographing apparatus according to the second embodiment is the same as the configuration shown in FIG. 1, and hence a description thereof is omitted here. Hereunder, only differences between the processing of the second embodiment with respect to the processing of the first embodiment are described.

According to the first embodiment, with respect to photometric calculation, the luminance values of the small blocks are determined using a mean luminance value. In this regard, a configuration may be adopted in which weights are assigned at required places. In a fundus oculi image, blood vessels are concentrated at an optic disc portion. Therefore, the optic disc portion is an important place with regard to diagnosis, and is also the brightest place in a fundus oculi image. Consequently, the exposure is liable to be bright at the optic disc portion. Therefore, an approach can be considered which focuses attention on the optic disc portion, and assigns a weight to the optic disc portion. The second embodiment follows this approach.

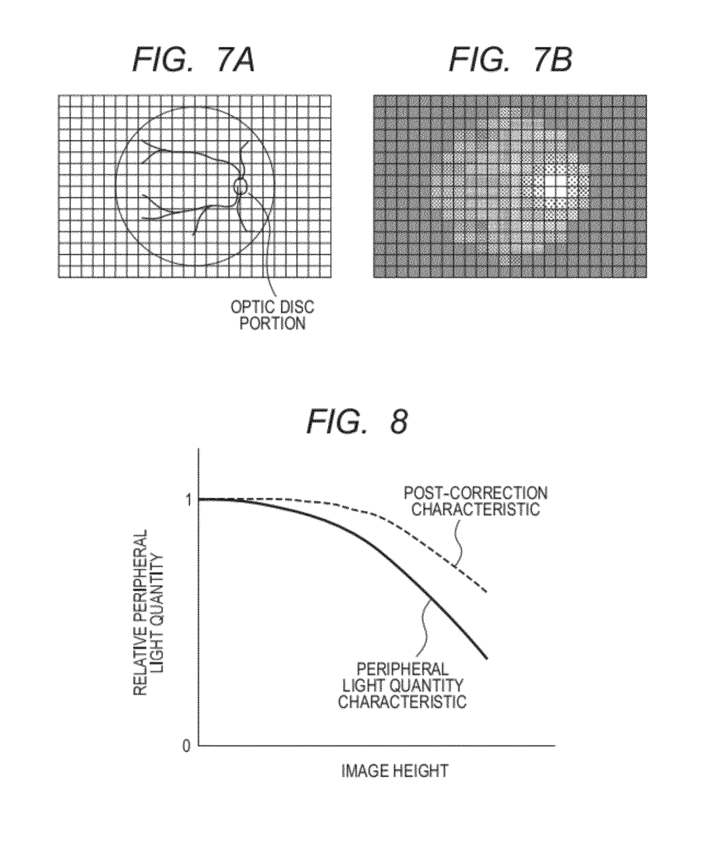

FIGS. 7A and 7B include views for describing photometry of a left and right eye. FIG. 7A illustrates a fundus oculi image of a right eye. For the right eye, the optic disc portion is projected on the right side. By focusing attention on the optic disc portion and assigning a high weighting thereto, automatic light adjustment in which exposure of the optic disc portion is not omitted can be realized. FIG. 7B illustrates the manner in which weights are assigned. In FIG. 7B, the brighter that a block appears in the figure, the higher that the weighting of the block is.

In the case of the left eye, it is necessary to assign weights in a manner in which the left and right sides shown in FIG. 7B are reversed. When performing brightness correction with the computer 25, the brightness correction can be performed by changing the evaluation pattern for weighting in accordance with whether the eye is the right eye or the left eye based on information regarding the setting of the left/right eye detection switch 24 at the time of photographing. Thus, brightness correction can be performed so that an exposure of the optic disc portion is not too bright.

### Third Embodiment

Next, a third embodiment of the present invention is described. The configuration of an ophthalmologic photographing apparatus according to the third embodiment is the same as the configuration shown in FIG. 1, and hence a description thereof is omitted here. Hereunder, only differences between the processing of the third embodiment with respect to the processing of the first embodiment are described.

A fundus camera performs photographing by illuminating a fundus oculi, which is a sphere, with a photographing light. Since the light distribution characteristics of the photographing light also exert an influence, a decrease in a peripheral light intensity is liable to occur. FIG. 8 is a view for describing vignetting correction according to the third embodiment. A solid line in FIG. 8 shows the characteristics of the actual peripheral light intensity, and illustrates how the light intensity changes in accordance with the image height when taking the optical axis center as 0. In FIG. 8, the relative light intensity is plotted in a case in which the light intensity at the center is taken as 1.

To improve a peripheral light intensity characteristic, image correction can be applied so that, in accordance with the peripheral light intensity characteristic, the brightness increases in accordance with an increase in the proximity to the periphery of the image. This kind of vignetting correction can be performed when the computer 25 performs development processing. The dashed line in FIG. 8 illustrates an example of the characteristics after vignetting correction is performed.

When the computer 25 performs brightness correction based on thumbnail image data of the digital camera 9, vignetting correction is not applied to the thumbnail image data that is generated at the digital camera 9. Accordingly, when the computer 25 performs brightness correction based on thumbnail image data, in some cases an excessive correction is performed. Hence, the computer 25 can prevent an excessive correction by correcting a target value of the brightness correction is accordance with a correction amount of the peripheral light intensity.

Further, when a state of alignment in a fundus oculi image is poor, flare light may enter a peripheral portion of the image. If vignetting correction is performed in a state in which the flare light remains in the image, the flare will be even more conspicuous. In such a case, the computer 25 may change the correction amount of the vignetting correction in accordance with the level of the flare light. When the correction amount of the vignetting correction is changed, the computer 25 can prevent excessive correction in the brightness correction processing by correcting the target value of the brightness correction processing in accordance with the changed correction amount.

### Fourth Embodiment

Next, a fourth embodiment of the present invention is described. According to the first embodiment, a case was described in which the present invention is applied to a non-mydriatic fundus camera. However, the present invention can also be applied to a mydriatic fundus camera, and the fourth embodiment describes such an example. In this case, an example of a hybrid fundus camera that can switch between mydriatic photographing and non-mydriatic photographing is described as the fourth embodiment.

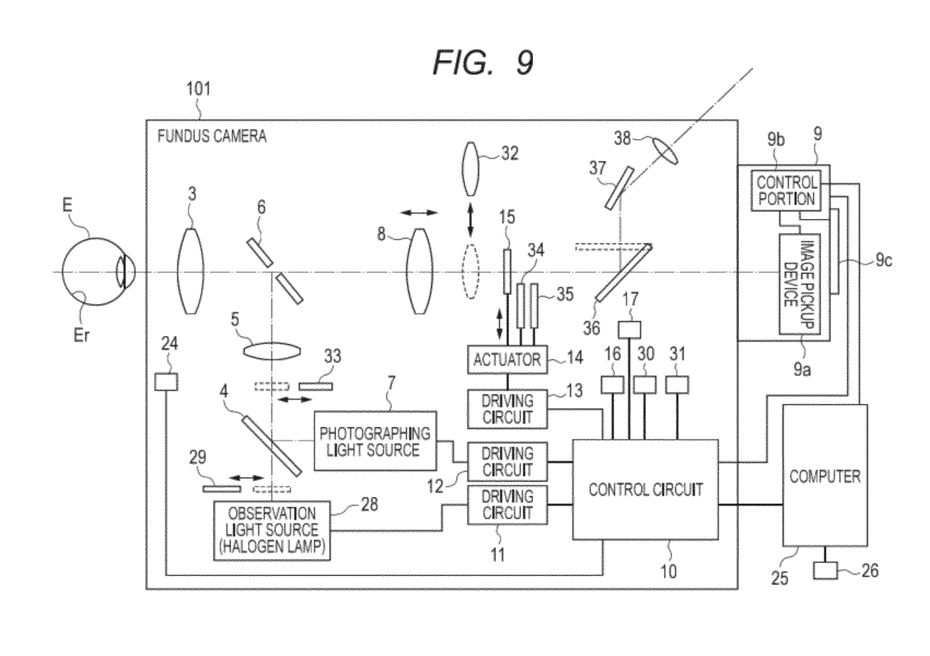

FIG. 9 is a view that illustrates a configuration of an ophthalmologic photographing apparatus according to the fourth embodiment. In FIG. 9, components that are the same as in the first embodiment are denoted by the same reference symbols, and a description of those components is omitted below. Further, the components denoted by reference numerals 18, 19, 21 and 23 in the first embodiment that do not directly relate to the description of the fourth embodiment are omitted from the configuration illustrated in FIG. 9. Hereunder, the configuration of a fundus camera 101 according to the fourth embodiment is described using FIG. 9.

A halogen lamp 28 is an observation light source that is different to the infrared LED of the observation light source 2 of the first embodiment. When emitting light, the halogen lamp 28 outputs a visible light component and an infrared light component simultaneously. A visible light cut-off filter 29 can be inserted into or withdrawn from the optical path of the illumination optical system at a time of observation. At a time of non-mydriatic observation, the visible light cut-off filter 29 is inserted into the optical path, and observation illumination is performed using only an infrared light component. At a time of mydriatic observation, the visible light cut-off filter 29 is withdrawn to outside the optical path, and observation illumination is performed by means of a visible light component and an infrared light component.

Switches 30 and 31 are connected to the control circuit 10. The switch 30 is used to switch between a mydriatic photographing mode and a non-mydriatic photographing mode. The switch 31 is used to switch the photographing mode. In this case, the term “photographing mode” refers to an optical photographing mode in which various kinds of optical filters are inserted into or withdrawn from the illumination optical system or the photographing optical system when photographing. According to the fourth embodiment, examples of photographing modes include, in addition to a normal color photographing mode, a red-free photographing mode and a fluorescence photographing mode. Other photographing modes include a cobalt photographing mode and an ICG photographing mode, and a configuration may be adopted in which these modes can be arbitrarily selected.

A mydriasis optical system 32 is configured so as to be inserted into the photographing optical path in the mydriatic photographing mode and to be withdrawn from the photographing optical path in the non-mydriatic photographing mode. Generally, for mydriatic photographing, a photographing angle of view is required that is wider than in non-mydriatic photographing. The mydriasis optical system 32 is an optical system for widening the photographing angle of view in the mydriatic photographing mode.

An exciter (excitation light) filter for fluorescence photographing 33 is inserted into a photographing illumination system when photographing in the fluorescence photographing mode. A barrier filter for fluorescence photographing 34 is inserted into the illumination optical path when photographing in the fluorescence photographing mode. A filter for red-free photographing 35 is inserted into the photographing optical path when photographing in the red-free photographing mode. A quick-return mirror 36 is disposed at a position indicated by a solid line in FIG. 9 that is in the photographing optical path at a time of observation in the mydriatic photographing mode. The quick-return mirror 36 reflects a visible light component upward. The visible light component that is reflected upward is guided by a mirror 37 and a viewfinder optical system 38 to constitute an optical viewfinder.

The quick-return mirror 36 includes a dichroic mirror that reflects visible light and transmits infrared light. The quick-return mirror 36 retracts to a position indicated by a dotted line in FIG. 9 when photographing in the mydriatic photographing mode, and thus a visible light component of reflected light from the fundus oculi is guided to the image pickup device 9a of the digital camera 9 when photographing. When the exposure for photographing ends, the quick-return mirror 36 returns to the position indicated by the solid line in FIG. 9.

In the non-mydriatic photographing mode, the quick-return mirror 36 is in a state in which the quick-return mirror 36 is retracted to the position indicated by the dotted line in FIG. 9, regardless of whether observation or photographing is being performed.

According to the above described configuration, in the non-mydriatic photographing mode the visible light cut-off filter 29 enters the illumination optical path so that observation illumination is performed by means of only an infrared light component. The mydriasis optical system 32 and the quick-return mirror 36 are withdrawn to outside the optical path, and operations that are the same as the operations described in the first embodiment are performed.

In the mydriatic photographing mode, the visible light cut-off filter 29 that is in the front of the halogen lamp 28 is withdrawn at the time of observation, and the mydriasis optical system 32 and the quick-return mirror 36 are inserted into the optical path. In this state, while the examiner is performing visible light observation of the fundus oculi of the eye to be examined by means of the optical viewfinder, an infrared light component passes through the quick-return mirror 36 that has dichroic mirror characteristics, and is guided onto the image pickup device 9a of the digital camera 9. Therefore, infrared photometry is enabled in a similar manner to the non-mydriatic photographing mode.

By taking into account the transmittance of the quick-return mirror 36 and differences in the optical system arising from insertion of the mydriasis optical system 32, it is possible to realize an automatic light adjustment function in the mydriatic photographing mode also that is the same as in the non-mydriatic photographing mode as described according to the first embodiment. As described in the foregoing, there is a difference in the photographing angle of view between the mydriatic photographing mode and the non-mydriatic photographing mode, with the photographing angle of view being wider in the mydriatic photographing mode. Therefore, it is necessary to set the evaluation range of brightness correction processing performed by the computer 25 for image data photographed in the mydriatic photographing mode to a wider range than for image data photographed in the non-mydriatic photographing mode.

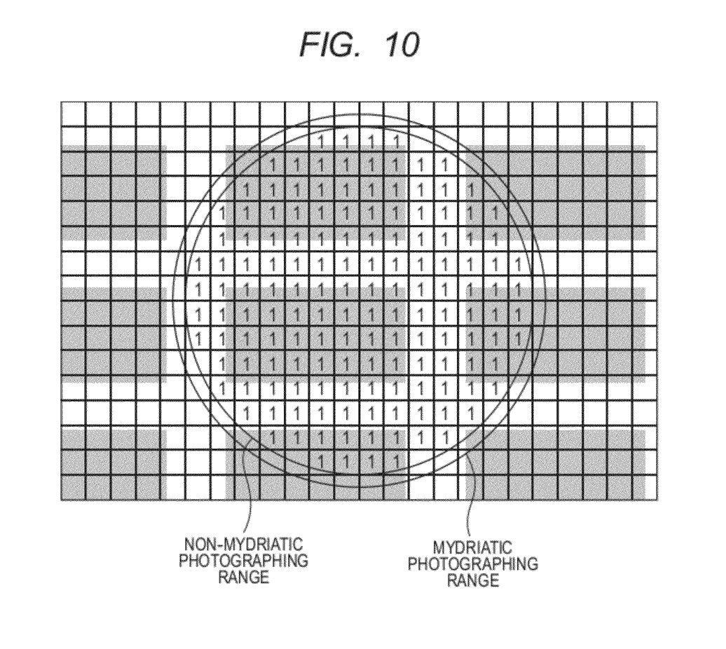

FIG. 10 is a view for describing photometry in a mydriatic photographing state according to the fourth embodiment. A circle on the inner side in FIG. 10 indicates the photographing range in the non-mydriatic photographing mode, and a circle on the outer side indicates the photographing range in the mydriatic photographing mode. Since a wider range is photographed at the time of mydriatic photographing, the number of blocks used for evaluation when performing brightness correction increases by a corresponding amount. Blocks that contain the numeral “1” in FIG. 10 are the blocks used for brightness correction when performing mydriatic photographing.

The target value of brightness correction also changes according to the photographing mode. For example, in the fluorescence photographing mode, a fluorescent agent is intravenously injected into the examinee, and a process in which the fluorescent agent flows into a blood vessel is photographed. The exciter filter for fluorescence photographing 33 is inserted into the illumination optical path in the fluorescence photographing mode. When the fluorescent agent is irradiated with an excitation light that passes through the exciter filter for fluorescence photographing 33, the fluorescent agent emits fluorescence of a wavelength that is shifted from the excitation light wavelength. Fluorescence photographing is performed by guiding only the fluorescence component to the image pickup device 9a by means of the barrier filter for fluorescence photographing that is inserted into the photographing optical path. Since the brightness of the photographed image data changes according to the amount of fluorescent agent that flows in the blood vessel of the fundus oculi, it is difficult to adjust the photographed image data to a predetermined brightness. Therefore, in the fluorescence photographing mode, the automatic light adjustment function is automatically turned off, and photographing is performed using a fixed photographing light intensity. Furthermore, brightness correction of photographed image data is not performed.

Further, in the red-free photographing mode in which photographing is performed by increasing the contrast of blood vessels, a filter for red-free photographing that allows only wavelengths in which the contrast of blood vessels is enhanced (mainly wavelengths of a green component) to pass therethrough is inserted into the photographing optical path. Therefore, the relationship between an irradiated light intensity at the time of photographing and a light intensity that is reflected back onto the image pickup device 9a is different to when performing normal color photographing. Further, in order to enhance the contrast of blood vessels, the ideal brightness of the image data will also between color photographing and the red-free photographing mode. Consequently, the target value of brightness correction of the photographed image data will also change between color photographing and the red-free photographing mode.

As described above, according to the fourth embodiment there is the advantageous effect that appropriate automatic light adjustment can be performed in accordance with differences between the mydriatic photographing mode and the non-mydriatic photographing mode, and the automatic light adjustment can be appropriately performed in various photographing modes.

According to the above described embodiments, an ophthalmologic photographing apparatus can be provided that includes a light adjustment function that is not dependent on racial or individual differences and that can reflect a preference of a photographer with regard to exposure. Note that the present invention is not limited to the contents described in the foregoing embodiments, and various changes are possible without departing from the scope of the present invention as defined in the claims.

### Other Embodiments

The present invention can also be realized by supplying software (a program) for realizing the functions of the above embodiments to a system or an apparatus via a network or via various storage media, and having a computer (or a central processing unit (CPU) or a micro processing unit (MPU)) of the system or apparatus read and execute the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-194718, filed Aug. 31, 2010, which is hereby incorporated by reference herein in its entirety.

What is claimed is:

1. An image processing apparatus for connection with an image pickup apparatus having a first light source for observing an object, a second light source for photographing the object and an image pickup unit, wherein the image pick up apparatus determines a light intensity of the second light source based on first image data obtained by illuminating an eye to be examined through the first light source and by forming an image on the image pickup unit using light reflected from the eye to be examined, the image processing apparatus comprising:
an analysis unit configured to analyze second image data obtained by illuminating the eye to be examined through the second light source with the light intensity determined by the image pickup apparatus and by forming an image on the image pickup unit using light reflected from the eye to be examined; and
a control unit configured to control brightness correction processing with respect to the second image data based on an analysis result obtained by the analysis unit and information relating to a time of photographing the second image data.

2. The image processing apparatus according to claim 1, wherein:
the analysis unit calculates luminance information of the second image data by splitting the second image data into predetermined blocks and determining a mean luminance of each block; and
the control unit controls the brightness correction processing with respect to the second image data in accordance with the calculated luminance information.

3. The image processing apparatus according to claim 2, wherein:
the analysis unit calculates luminance information of the second image data by performing a predetermined weighting operation with respect to a mean luminance of the respective blocks; and
the control unit controls the brightness correction processing with respect to the second image data in accordance with the luminance information.

4. The image processing apparatus according to claim 1, wherein:
the information relating to the time of photographing includes information showing whether a function of an automatic light adjustment unit of the image pickup apparatus is set to an enabled state or set to a disabled state at a time of photographing the second image data; and
the control unit switches control of the brightness correction processing with respect to the second image data in accordance with whether the function of the automatic light adjustment unit is set to an enabled state or set to a disabled state.

5. The image processing apparatus according to claim 1, wherein:
the information relating to the time of photographing includes diseased eye information that shows a disease of the eye to be examined; and
the control unit switches control of the brightness correction processing with respect to the second image data in accordance with the diseased eye information of the eye to be examined.

6. The image processing apparatus according to claim 1, wherein:
the information relating to the time of photographing can include information showing a photographing mode of the image pickup apparatus; and
the analysis unit switches an analyzing range of the second image data in accordance with whether or not the information relating to the time of photographing includes the information showing a photographing mode of the image pickup apparatus.

7. The image processing apparatus according to claim 6, wherein the photographing mode includes at least one of an anterior ocular segment photographing mode, a small pupil photographing mode, a variable magnification photographing mode, and a mydriatic photographing mode.

8. The image processing apparatus according to claim 1, wherein:
the information relating to the time of photographing includes information showing whether the eye to be examined is a left eye or a right eye; and
the control unit switches control of the brightness correction processing in accordance with whether the eye to be examined is a left eye or a right eye.

9. The image processing apparatus according to claim 1, further comprising a correction unit configured to correct a peripheral light intensity of the second image data, wherein the control unit switches control of the brightness correction processing with respect to the second image data in accordance with a correction amount of the peripheral light intensity performed by the correction unit.

10. An image processing apparatus, comprising:
a setting unit configured to set a partial range of an image of an eye to be examined based on a photographing mode of the image;
a calculation unit configured to calculate a luminance value of the partial range; and
a correction unit configured to correct a brightness of the image based on the luminance value and the photographing mode.

11. An image processing method, comprising:
setting a partial range of an image of an eye to be examined based on a photographing mode of the image;
calculating a luminance value of the partial range; and
correcting a brightness of the image based on the luminance value and the photographing mode.

12. A non-transitory computer-readable storage medium storing a computer program for causing a computer to execute the method according to claim 11.

13. An ophthalmic system, comprising:
a setting unit configured to set a partial range of an image of an eye to be examined based on a photographing mode of the image;
a calculation unit configured to calculate a luminance value of the partial range; and
a correction unit configured to correct a brightness of the image based on the luminance value and the photographing mode.
